(19) United States
(12) Patent Application Publication
(10) Pub. No.: US 2005/0075651 A1
(43) Pub. Date: Apr. 7, 2005

(54) Safe cut callus remover

(76) Inventor: Alvaro Ernesto Ortiz, Miami, FL (US)

Correspondence Address: Alvaro Ernesto Ortiz, 402 Nw 43 PI, Miami, FL, 33126, US

(21) Appl. No.: 10/877,219

(22) Filed: Jun. 25, 2004

Related U.S. Application Data
(60) Provisional application No. 60/483,062, filed on Jun. 30, 2003.

(51) Int. Cl. A61B 17/32

(52) U.S. Cl. 606/131; 606/184

(57) ABSTRACT

Skin care hand held device and method for caring for human hands and feet useful for safely removing calluses, cuticles, warts and dead cells from human hands and feet, applying a circular, rounded cutting blade to the human skin is disclosed. The present invention relates to a cylindrical scalpel comprising a tube where the tip cut at a 90-degree angle is sharpened to form a curved circular blade integrated with the shank. The blade portion is formed by the inner wall of the tube and the external wall which is formed as to have a curved shape on the outside rim at the tip of the tube, the hollow bore allows access for application of liquid or gaseous additives or vacuum or other types or energy to be applied directly through the tube. Methods for using the foot care device are also disclosed.

Priority is claimed on provisional application No. 60483062 entitled SAFE CUT CALLUS REMOVER, filed on Jun. 30, 2003 by Alvaro E. Ortiz a citizen of The United States Of America, residing at 402 NW 43 Pl. Miami, Fla. 33126 USA.

### BACKGROUND OF THE INVENTION

The present application relates generally to skin care products and methods for caring for human feet and hands. More specifically, it relates to skin care products and methods for removing calluses, corns, cuticles and the like from human feet and hands. Most specifically, it relates to cutting skin care products and methods for removing calluses, corns and the like from human feet and hands operated by applying by hand a cutting blade to the human skin. It is known that skin built up on the foot such as calluses and corns are a recurring problem to human feet and hands having been subjected to prolonged friction and pressure. Currently available treatments such as medicated pads and other devices for the removal of such hardenings of skin have been found not to provide prompt or adequate relief to the feet and hands. One particular prior art design for removing dead skin, calluses and other formations from the foot and stimulate its blood circulation is described in U.S. Pat. No. 4,246,914 to Keyser. This particular patent discloses an elongated bar having an abraded surface and a smooth surface. In use, the bar is placed horizontally, e.g., on the floor, vis-à-vis the seated user, who places his right foot firmly and supportively on the smooth surface of the bar so that the bar will remain stationary on the floor.

The user then draws his left foot across the abraded surface which when drawn with adequately applied pedal pressure across the abraded surface, relieves calluses, corns, dead skin, etc. developed under the foot sole.

While this prior art device was successful in providing some relief for sufferers of foot calluses and corns, using such device and other currently available treatments for such calluses, etc. such as, for example, a pumice stone have all proven to work partially, abrading both, dead and living cells, making impossible the removal of all the dead cells, before bleeding or discomfort occur. Thus, there is a need for devices and methods for caring for human feet and hands which are safe to use to allow a complete removal of calluses, corns and skin buildup on human hands and feet. Such devices and methods should smooth the rough skin on the bottom, top, sides and in between the toes of the feet. Such devices and methods should reduce calluses and provide general foot maintenance. Such devices and methods should have non-allergenic properties. Such devices and methods should be safe to be operated by the user. Such devices and methods should assist in the alleviation of foot discomfort due to skin build-up and calluses.

### SUMMARY OF THE INVENTION

The object of the invention is to overcome the aforementioned disadvantages of the prior art devices.

It is one object of the present invention to provide devices and methods for safely removing the rough skin on the bottom, top, sides and in between the toes of the feet.

It is another object of the present invention to provide devices and methods which reduce calluses and provides general foot maintenance.

It is yet another object of the present invention to provide devices and methods which produces a renovating effect to the feet and hands when they are cleaned by this device.

It is still another object of the present invention to provide devices and methods that has non-allergenic properties.

It is another object of the present invention to provide devices and methods that are resistant to mildew.

It is yet another object of the present invention to provide cutting devices and methods which increase the safety and comfort of a user during use.

It is still another object of the present invention to provide a device which aids and assists in the alleviation of foot discomfort due to skin build-up and calluses.

In accordance with these and further objects, one aspect of the present application includes a device for at least partially alleviating foot discomfort due to skin build-up, such as, calluses, the device comprising a tube where the tip cut at a 90 degree angle is sharpened to form a curved circular blade integrated with the shank. The blade portion has a calibrated curved inward cutting edge with a radius formed so as to have a ratio of the partial thickness thereof total thickness thereof within a range of from 5 to 1000%.

Yet another aspect of the present application includes a method for removing skin from a human foot, the method comprising the steps of: wet and lather the foot with a thin layer of soap and water to soften the skin and so that the device can glide easily over the rough skin of the foot; and moving the blade over the outer surface of the foot in at least one of a plurality of possible motions while applying slight pressure with the hand.

Other objects and advantages of the application will be apparent from the following description, the accompanying drawings and the appended claims.

### BRIEF DESCRIPTION OF THE DRAWINGS

FIG. 1 is an elevation view of a first representative embodiment of one possible skin care device of the present invention, illustrates an alternative method for grasping the tube to operatively rub the cutting blade perpendicularly over the skin with downward upward movements as well as from side to side.

FIG. 2 is an elevation view of the of the embodiment of FIG. 1; illustrates another exemplary method for grasping the tube to operatively rub the blade over the skin with downward upward movements as well as from side to side.

FIG. 3 is an elevation view of the of the embodiment of FIG. 1; illustrates another exemplary method for grasping the tube to operatively rub the cutting blade over the skin of the foot.

FIG. 4 is an elevation view of one device of a series of possible embodiments of skin care devices of the present invention in this case using a gripping liner.

FIG. 5 is a cross-sectional view of a set of four possible embodiments of the present invention, illustrates four alternative forms for the blade formed on the tip of the tube of a limitless number of different possible shapes of one possible skin care device of the present invention.

FIG. 6 is a cross-sectional view of the of four possible embodiments of the present invention, illustrates an alternative method for adding additives positioning on the inside of the shank of the device a number of smaller diameter tubes as illustrated in each of the four possible embodiments of the present invention.

FIG. 7 is a cross-sectional enlarged view of a set of three possible embodiments of the present invention using three different curves to form the blade on the tip of the tube after sharpening of a limitless number of different possible curves of one possible skin care device of the present invention.

FIG. 8 is a cross-sectional enlarged view of three possible embodiments of the present invention, illustrates three different curves used to form the blade on the tip of the tube of a limitless number of different possible curves of one possible skin care device of the present invention.

FIG. 9 is an elevation view of six devices made with different diameters using surgical stainless steel tubes.

FIG. 10 is an elevation view of one device made with surgical stainless steel tube showing the shape of the tip of one embodiment of the present invention.

FIG. 11 is an elevation view of an alternative method for grasping the device with a gripping liner of one embodiment of the present invention

FIG. 12 is an elevation view of one device of a series of possible embodiments of skin care devices of the present invention in this case a gripping liner connected to a pump or vacuum machine using the flexible tubing connected to the back of the liner.

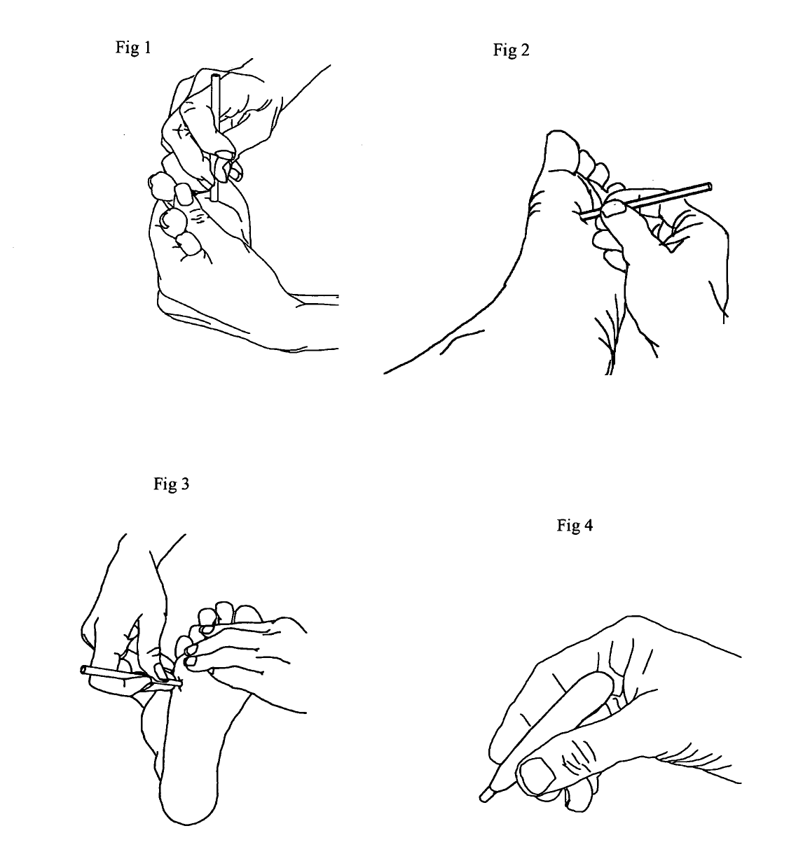

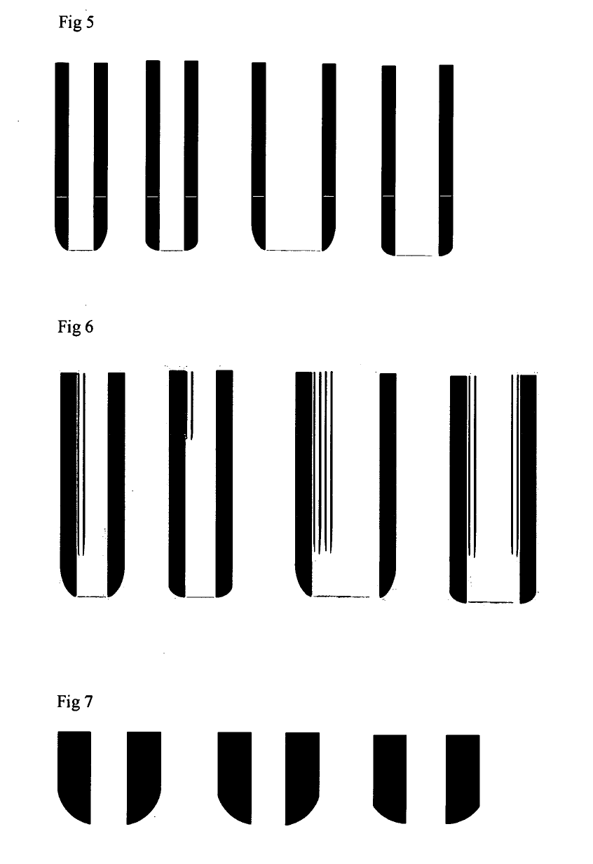

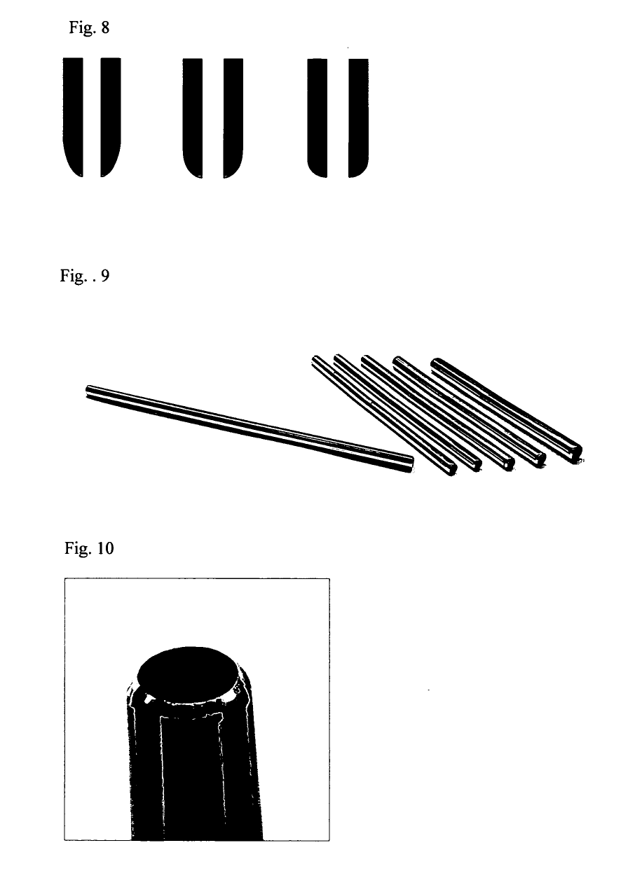

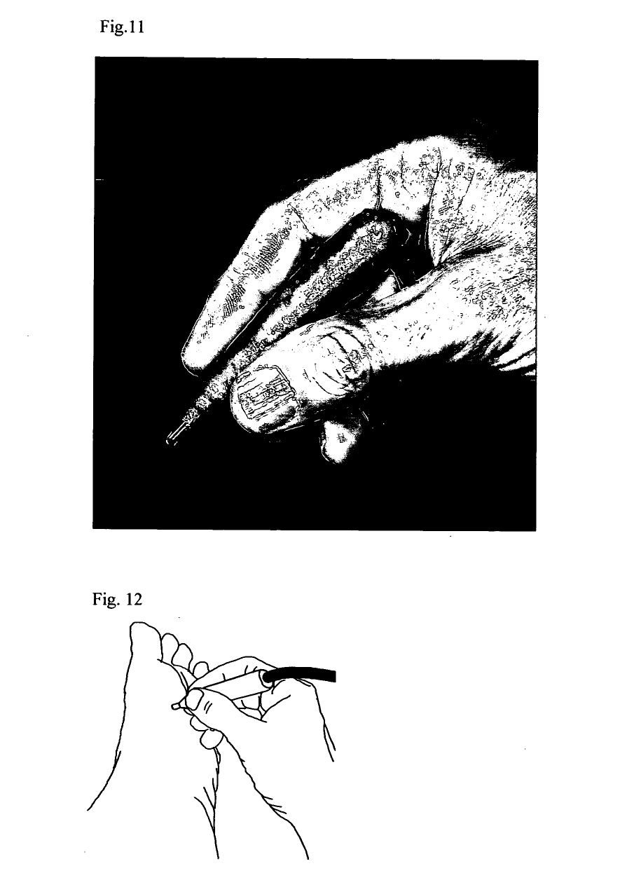

### DETAILED DESCRIPTION

The present invention is a skin care device useful for the exfoliation of the flaking top layer of skin on the foot, the exfoliation and reduction of calluses on the foot and for general skin care maintenance. These exfoliations become necessary when the skin build-up or callus produces pain and/or discomfort while wearing shoes, walking, preparing for sleep and other normal everyday activities. It is believed that many people suffer from the effects of this skin build-up and therefore normal and daily activities performed are done so with, in some cases, at least some discomfort. The present invention is designed to assist in the alleviation of this discomfort.

The present invention relates to a cylindrical scalpel for selectively abrading and removing only dead skin cells leaving untouched the adjacent living cells comprising a tube where the tip cut at a 90 degree angle is sharpened to form a curved circular blade integrated with the shank.

The blade portion is formed by the inner wall of the tube and the external wall which is shaped as to have a calibrated inward curved cutting edge predetermined for selectively abrading and removing rigid dead skin cells, leaving untouched the pliable adjacent living cells.

This blade is designed so that immediately after it has removed all the rigid callus and rough skin and finds soft, pliable skin, the angle of incidence of the cutting blade as it passes over this layer of new flexible skin becomes too great, as result, the cutting edge losses its grip, it stops cutting and glides over this layer of healthy skin, thereby letting the user know the dead, rough skin was removed and no more passes are needed.

The blade is formed by removing material from the outer wall. This is preferably accomplished by means of a lathe or running a grinding head against the outside wall of the shaft to form the desired shape or by means of an electrochemical process or by casting on a mold were the material to form the cutting edge is poured in liquid state or pressed into this mold or by deposition of material in the form of crystals or any other means and processes that are capable of producing such device.

The tube preferably has a length sufficiently long to serve as handle to be grasped between the thumb and at least two fingers as shown in FIG. 1

Referring to FIG. 1, an exemplary method for removing callus using the circular blade. Initially, the tubular device is grasped by a hand H and positioned over a patient's foot as shown in FIG. 1. The blade is manipulated until the shaft is substantially perpendicular to the patient's skin at the point of intended removal. The blade 1 is then rubbed over the callus as shown in FIG. 1. Preferably, the blade will be rubbed over the callus or corn with sufficient pressure as to allow the removal of the superficial layer of skin.

Repeated passes of the device over the area of skin being treated remove the remaining layers of hard skin until the device finds soft pliable skin and the device glides easily without cutting, letting the user know the dead and rough skin was removed.

The gripping liner serves two main purposes, it provides a comfortable grip and connecting means using flexible tubing to connect the device to a pump or vacuum device or laser, ultrasound or electromagnetic generator providing simultaneous transmission of energy trough the main shank and injection of liquid or gaseous additives through independent smaller diameter tubes positioned inside the main shank of the tube.

The additives sprayed could cover the surface being cut providing lubrication or moisturizing quality or any other quality desired, steam, liquid nitrogen to freeze and make rigid the layer of skin being removed, anesthetic agents, disinfectants or any other additive with the desired properties or may be used as a washing fluid that may be helpful in using the device if the device is connected to a vacuum machine so that when the blade is applied by the user to the surface of the foot and the cutting blade is removing skin the vacuum interacts with the additive to wash and pick up trough the tube debris and scales of dead skin as is being removed.

While the devices and methods described herein constitute preferred embodiments of the invention, it is to be understood that the invention is not limited to these precise devices and methods and that changes may be made therein without departing from the scope of the invention which is defined in the appended claims.

1. A device for removing dead skin, calluses, corns alleviating skin discomfort, the device comprising:
a tubular structure having an upper and a bottom end sharpened to form a cutting edge.

2. The device of claim 1 further comprising:
at least one inward curved circular blade at the end of the tubular structure as cutting means for safely removing skin build up from a human foot or hand.

3. The device of claim 1 further comprising:
the tubular structure having sufficient length and diameter to make it suitable for a comfortable grip by the human hand.

4. The device of claim 1 further comprising:
a gripping liner were the tube is inserted into as gripping means for the device in claim 1

5. The device of claim 1 further comprising:
tubing as connecting means for the device in claim 1

6. The device of claim 1 wherein, the cutting means comprises:
a combination of a plurality of materials.

7. The device of claim 1 wherein the blade material is selected from the group comprising:
steel. stainless steel, iron, brass, bronze, nickel, aluminum, chrome, titanium, platinum, gold, silver, tantalum, tungsten, iridium, palladium, rhodium, ruthenium, osmium

8. The device of claim 1 wherein the blade materials, are selected from the group comprising:
plastics, synthetics, glass, carbon, aramids, crystals, ceramics, composites.

9. The device of claim 1 wherein additive ingredients are operatively positioned on the inner surface of the cutting blade

10. The device of claim 2 wherein the at least one cutting blade is operatively inserted on a liner or handle leaving the cutting tip of the device exposed to operatively connect the other end by means of flexible tubing to a pump or vacuum device or laser, ultrasound or electromagnetic or other types of generators providing simultaneous transmission of energy or matter trough the main shank and injection of liquid or gaseous additives through other independent smaller diameter tubes positioned inside the hollowed bore of the device.

11. The device of claim 1 wherein, the gripping means comprises a combination of a plurality of materials.

12. The device of claim 3 wherein the gripping means materials are selected a combination of a plurality of materials from the group comprising:
steel. stainless steel, iron, brass, bronze, nickel, aluminum, gold, chrome, titanium, platinum, silver, tantalum, tungsten, iridium, palladium, rhodium, ruthenium, osmium

13. The device of claim 3 wherein the gripping means materials, are selected from the group comprising:
plastics, synthetics, rubber, glass, carbon, aramids, crystals, ceramics, composites.

14. A method for removing skin from a human anatomy, the method comprising the steps of:
wet and lather the skin to soften it

15. The method of claim 14 further comprising the step
covering the skin with a thin layer of lubricant so that the blade can glide easily over the skin.

16. The method of claim 14 further comprising the step of:
disinfecting and moisturizing the area of skin treated to prevent infection
